(19) United States
(12) Patent Application Publication
(10) Pub. No.: US 2008/0319391 A1
(43) Pub. Date: Dec. 25, 2008

(54) Self powered enteral tube feeding device

(76) Inventor: Francis J. Jackson, La Mesa, CA (US)

Correspondence Address: John R. Ross, P.O. Box 2138, Del Mar, CA, 92014, US

(21) Appl. No.: 11/821,074

(22) Filed: Jun. 21, 2007

(51) Int. Cl. A61M 5/14

(52) U.S. Cl. 604/142; 604/404

(57) ABSTRACT

A self-powered, enteral tube feeding device. The device includes a fluid container and a removable lid for pressure sealing the container. The device also includes a fluid exit port and a flexible tube providing fluid communication from the exit port of the device to a through-the-abdominal-wall port of a patient. Fluid is forced out of the container and into the patient's stomach or small intestine with air pressure provided by a finger-operated flexible bulb-type pressurizer unit having two air check valves. The device is preferably operated by the user without assistance. In preferred embodiments the device also includes alarm features that provide a warning when the fluid level is approaching the exit port so that no air is pumped into the user's abdominal cavity. In one preferred embodiment these alarm features include a magnetic switch located below the fluid container and a magnet floating within the container vertically over the switch and on or near the surface of the fluid.

The present invention relates to enteral feeding devices and especially to devices for enteral feeding through the abdominal wall.

### BACKGROUND OF THE INVENTION

### Enteral Tube Feeding

Enteral feeding literally means using the gastrointestinal tract for the delivery of nutrients, which includes eating food, consuming oral supplements, medications, and all types of tube feeding. The routes of enteral tube feeding may be through the nose into the stomach, through the nose into the small intestine or through the abdominal wall into the small intestine.

Enteral tube feeding has become a widely used health care technology both in the hospital and community settings, and its increasing use is associated with a range of different routes and systems for delivery of nutrition.

### Gastric Feeding Tube

A gastric feeding tube, or “G-tube”, is a tube inserted through a small incision in the abdomen into the stomach and is used for long-term enteral nutrition. The most common type is the percutaneous endoscopic gastrostomy (PEG) tube. It is placed endoscopically: the patient is sedated, and an endoscope is passed through the mouth and esophagus into the stomach. The position of the endoscope can be visualized on the outside of the patient's abdomen because it contains a powerful light source. A needle is inserted through the abdomen, visualized within the stomach by the endoscope, and a suture passed through the needle is grasped by the endoscope and pulled up through the esophagus. The suture is then tied to the end of the PEG tube that will be external, and pulled back down through the esophagus, stomach, and out through abdominal wall. The insertion takes about 20 minutes. After the insertion, the abdominal wound must be covered with sterile dressings until it is healed (about a week). The tube is kept within the stomach by a balloon on its tip (which can be deflated to remove the tube).

Gastrostomy tubes can also be placed in “open” procedures through an incision with direct visualization of the stomach, as well as via laparoscope. Gastric tubes are suitable for long-term use: they last about six months, and can be replaced through an existing passage without an additional endoscopic procedure. The G-tube is useful where there is difficulty with swallowing because of neurologic or anatomic disorders (stroke, esophageal atresia, tracheoesophageal fistula), and to avoid the risk of aspiration pneumonia. It is also used when patients are malnourished and cannot take enough food by mouth to maintain their weight. They also can be used in “reverse” to drain stomach contents.

### Jejunostomy Tube

A jejunostomy tube is similar to a gastric tube, though generally has a finer bore and smaller diameter, and is surgically inserted into the jejunum rather than the stomach. They are used when the upper gastrointestinal tract must be bypassed completely, and can be used as soon as 12 hours after surgery. This type of tube is usually used for people who have stomach ulcers.

These small bore tubes are prone to clogging, particularly with some medications and if not flushed as directed. Feeding through these tubes are generally commercially prepared to provide adequate nutrition and to not result in clogging when used with a pump or with drip feedings.

### Growth of Home Enteral Tube Feeding

Enteral tube feeding in the community has increased considerably in recent years. One study showed a 26% growth in adults in the community on home enteral tube feeding from 1998 to 1999 and an average growth rate prior to 1998 has been estimated at 20-25% per year. Several reasons have contributed to the rapid growth of enteral tube feeding outside the hospital including reduction in the number of hospital beds, developments in artificial nutrition, higher proportion of elderly subjects in the population, promotion and marketing of home enteral tube feeding devices by commercial companies and increased awareness of therapeutic nutrition.

### Starting Home Enteral Tube Feeding

Difficulty in swallowing, or the inability to swallow at all, is the most common primary reason for initiating enteral tube feeding. The difficulty or inability may result from accident, multiple sclerosis, motor neurone disease, various types of cancer and cerebral trauma. Other common reasons for enteral tube feeding include aiming to improve or maintain nutritional status, malabsorption and anorexia.

Enteral tube feeding is usually initiated in a hospital and that the patient is subsequently discharged into the community. However, more doctors are now referring electively for home enteral tube feeding, and patients are having through-the-abdominal-wall tubes sited as day-patients. However, a number of complications post insertion have been recently identified, so overnight admission is recommended.

### Gastrostomy Tubes

In the community the through-the-abdominal-wall tubes are the most common and easiest to manage. Tubes are placed usually under local anaesthetic, with a small incision made in the abdominal wall, and the tube is inserted with the help of an endoscope. These tubes vary in size, and normally last for 18 months to 2 years when it may be advisable to replace them by repeat endoscopy. When the tubes are no longer required, they may be removed or they can be cut and allowed to pass naturally, although this remains controversial. The tubes are typically extended through the abdominal wall into the jejunum which is the upper portion of the small intestine that extends from the duodenum to the ileum. The surgical creation of this permanent opening is called jejunostomy. The opening so established is also called a jejunostomy.

One of the most important things patients and care givers need to be taught is caring for the feeding tube correctly. This includes flushing the tube with water immediately after any feed or medication has been administered via it. The most common cause of blocked tubes is leaving too long a time between feeding and flushing. Should a tube block, there are a variety of different tactics which may help unblock it including flushing with fizzy drinks, pineapple juice or sodium bicarbonate, whilst manipulating the tube between the fingers. There are also enzyme preparations which can be used in extreme cases. Inserting a sharp object down the tube to remove a blockage is discouraged.

Typically an external fixation plate on the tube prevents the tube from being drawn into the gastro-intestinal tract. It needs to be turned 90° daily to allow the site to be inspected and cleaned. The tube should also be rotated and pushed slightly to prevent it from becoming adherent to the gastric mucosa of the abdominal wall, which is known as bumper syndrome.

Balloon retained gastrostomy tubes are increasingly becoming the choice of feeding for long term community feeders, as they can be replaced more easily and by the patient or care-giver. Generally, these are only sited when an opening in the abdominal wall has already been formed by prior tube insertion. A balloon inflated by sterile water holds the tube in place, which needs re-inflating about once a week; although some more modern tubes may be checked less frequently. Balloon gastrostomy tubes need replacing every 2-3 months and arrangements should be made for further supplies of spares. Prompt replacement is crucial to avoid closure of the stoma. Balloon gastrostomy tubes are particularly favorable in those patients for whom the endoscopy procedure is traumatic or the practicality of transporting the patient to hospital is hard.

There are complications associated with through-the-abominable-wall feeding: Soreness at the stoma site is not uncommon, and this may involve redness, inflammation and sometimes smelly discharge. Some neurological patients have reported feeling very poorly for a few days following the procedure. Blocked tubes are a common problem, though good practice of caring for the tube should prevent this; unfortunately in the nursing home environment, with frequently changing staff and bank nurses, the incidence of blocked tubes is quite high. Intolerance of feed may be reported, or may be helped by changing the feed, on which the dietitian will advise.

### Prior Art Enteral Tube Feeding Devices

A wide variety of enteral tube feeding devices are commercially available from suppliers such as the Ross division of Abbot Labortories. At the writing of this specification, a list of these devices can be found on-line at “www.ross.com”. Kits are available from Ross with jejunostomy devices to provide a port through the abdominal wall into the upper regions of the small intestine. The simplest feeding device is the gravity feed nutrition bag. These devices are used similarly to intravenous bags except food, usually in ground up fluid form with viscosity of motor oil, is feed by gravity from a suspended bag through a plastic tube through the port in the abdominal wall into the upper region of the small intestine. With the bag at a few feet above the patient the process is slow taking more than an hour for one feeding, but users have learned that the process can be speeded up by increasing the relative height of the bag. Also many enteral pumps are available from Ross and others for pumping the liquefied food through the abdominal port into the small intestine. These pumps include safety devices and flow rate monitors and indicators. Pumps are generally fairly expensive and require a standard power source, although some are battery powered.

What is needed is a simple, quick, inexpensive, reusable, self-powered enteral tube feeding device especially for home use.

### SUMMARY OF THE INVENTION

The present invention provides a self-powered, enteral tube feeding device. The device includes a fluid container and a removable lid for pressure sealing the container. The device also includes a fluid exit port and a flexible tube providing fluid communication from the exit port of the device to a through-the-abdominal-wall port of a patient. Fluid is forced out of the container and into the patient's stomach or small intestine with air pressure provided by a finger-operated flexible bulb-type pressurizer unit having two air check valves. The device is easily operated by the user without assistance. In preferred embodiments the device also includes alarm features that provide a warning when the fluid level is approaching the exit port so that the user, or caregiver, will become aware that the fluid level is low and can avoid the flow of air into the stomach. In one preferred embodiment these alarm features include a magnetic switch located below the fluid container and a magnet floating within the container vertically over the switch and on or near the surface of the fluid.

### BRIEF DESCRIPTION OF THE DRAWINGS

FIG. 1 is a drawing of a preferred embodiment of the present invention.

FIG. 2 is a drawing of a second preferred with alarm devices.

FIG. 3 is a drawing of a easily portable preferred embodiment.

FIG. 4 shows a portion of a preferred warning element.

### DETAILED DESCRIPTION OF PREFERRED EMBODIMENTS

### First Preferred Embodiment

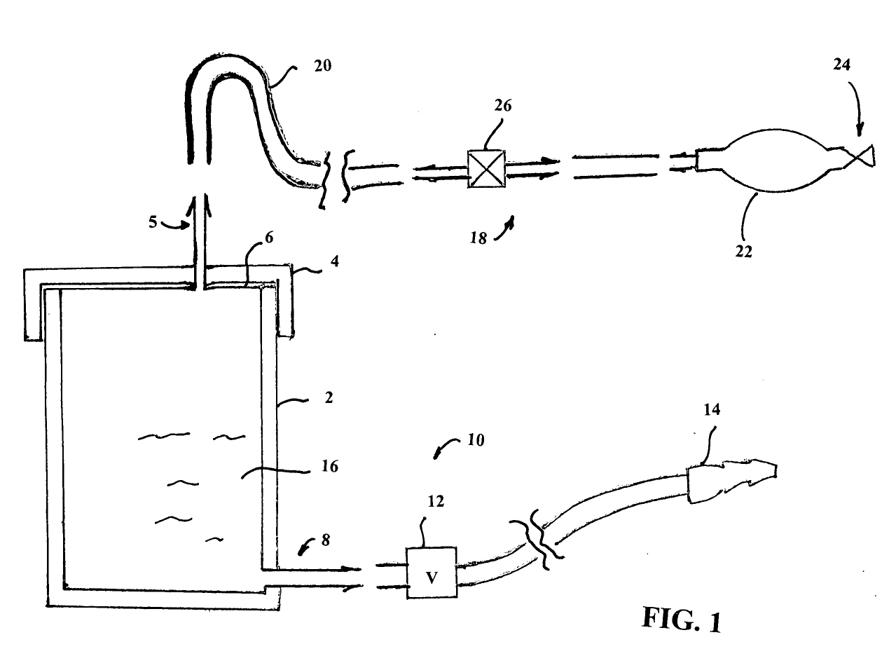

FIG. 1 is a drawing of a first preferred embodiment of the present invention. This embodiment includes a clear plastic fluid container 2, a removable lid 4 and rubbery sealing pad 6 for pressure sealing the container. The clear plastic provides an unobstructed view of the liquid food level, and provides a clear view of cleanliness when washed. This preferred embodiment includes a three-tab bayonet type locking seal. This type of seal works well for sealing wide openings in containers, but many other sealing features could be substituted. (An example of a bayonet type seal is described in U.S. Pat. No. 7,150,378 which is incorporated herein by reference.) Container 2 includes a fluid exit port 8 and flexible tube 10 provides fluid communication from the exit port of the device to a through-the-abdominal-wall port of a patient. Tube 10 includes stop valve 12 which is an inexpensive tube clamp type stop valve (Part No. K9325 available from IMED with offices in New York, N.Y.) and a tube connector part 14 for making a connection with the entry port of the user's tube feeding equipment implanted in his abdominal wall. This part is available from the supplier of the implanted parts. Container 2 contains fluid nutrition 16. The fluid is forced out of the container and into the patient's small intestine with air pressure provided by a finger-operated flexible bulb-type pressurizer unit 18. This unit includes two air check valves 24 & 26, plastic tubing 20 and bulb unit 22. A preferred bulb unit is part No. PM80BULB available from Safety Store with offices in Charlottesville, Va. This unit contains one of the two check valves, i.e. valve 24. The other check valve is Part No. 2774K11 available from McMaster with offices in Los Angeles, Calif., and is shown at 26. Normal operation of pressurizer unit 18 produces a pressure in container 2 of about 1 psi which is the recommended pressure for transfer of a meal in about 5 to 10 minutes. Rapid firm pumping can produces pressures of about 5 psi but this much pressure is not recommended since it would result in extremely rapid transfer of the fluid. Lid 4 contains pressurized air entry port 5 for connecting tube 20 of pressurizer unit 18. The device is easily operated by the user himself, but a care-giver could operate it. With the device shown in FIG. 1, care must be taken to be certain that the process is halted before the level of the fluid drops to as low as exit port 8 so that no substantial amount of air is pumped into the user's abdominal cavity. As explained above, the user or his caregiver is in control of the pumping operation. A small amount of air in the abdominal cavity typically would be no problem, except maybe to produce a belch. The reader should note that the preferred embodiment shown in FIG. 1 includes a gradually sloped bottom with a V-groove at the exit port. This assures that no air can enter the exit port until virtually all of the fluid has been forced out. This embodiment is a very inexpensive device for through the abdominal wall tube feeding. It has many advantaged over the gravity bag tube feeding units and expensive pump devices. For example, the time required for a typical meal is only about 7 to 10 minutes as compared to an hour or more. Also the present invention is very easily cleaned in a dish washer and can be used over and over indefinitely. Tube 10 can be cleaned by flushing. The unit requires no power except finger power. Also it is very portable.

### Second Preferred Embodiment with Warning Feature

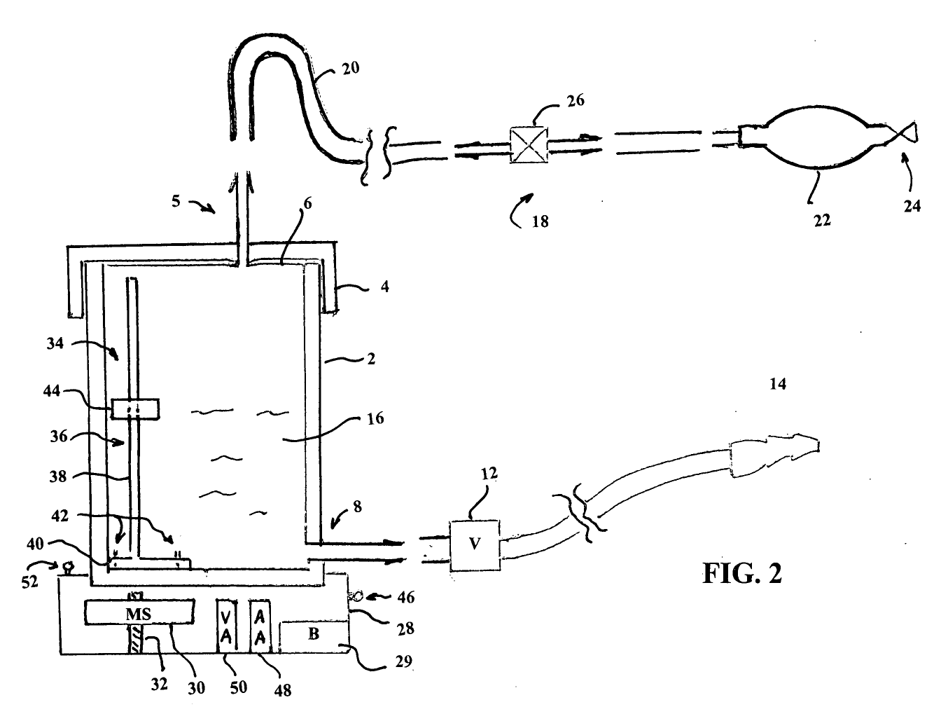

A second preferred embodiment of the present invention is shown in FIG. 2. This embodiment is the same as the one shown in FIG. 1 except it also contains warning elements most of which are contained in a separate fixture 28 with the container sitting on top. Inside fixture 28 is battery 29 and magnetic switch 30 which closes in the presence of a sufficient magnetic field. In this preferred embodiment fixture 28 is splash proof only and should not be immersed in water or cleaned in a dish washer. Magnetic switch 30 is positioned on threaded post 32 so its vertical position can be adjusted. Magnetic trigger unit 34 is positioned inside container 2. Unit 34 is comprised of stand 36 which is preferably made of stainless steel and includes thin cylindrical rod 38 and platform 40. Platform 40 includes two location holes which properly locate float unit 34 over magnetic switch 30 with two tabs 42 located on the bottom of container 2. Float unit 34 also comprises a magnet not shown contained in a lighter-than-water magnetic float element 44 having a center line hole so that magnetic float element 44 can slide down rod 38 as the fluid level in container 2 drops. When the level of fluid 16 is near the bottom of container 2, the magnet in float element 44 is near enough to magnetic switch 30 to cause the switch to close. The closing of switch 30 causes warning light 46 to flash, audio alarm 48 to alarm and vibration alarm 50 to vibrate. These warnings will remind the user or his care-giver to terminate the feeding before any air can enter the user's abdominal cavity. On-off switch 52 permits the user to turn off the alarms. Threaded post 32 permits the position of magnetic switch 30 to be adjusted to fine-tune its location relative to the magnetic float so that the alarms are energized at the proper fluid level.

This embodiment has all of the advantages of the first embodiment except it is somewhat more expensive to produce. The user if he wants is not required to use the warning parts since the container is comprised of clear plastic and he can see the fluid level. The container, lid and the parts of float unit 34 can be washed in the dishwasher.

### Simple Portable Unit

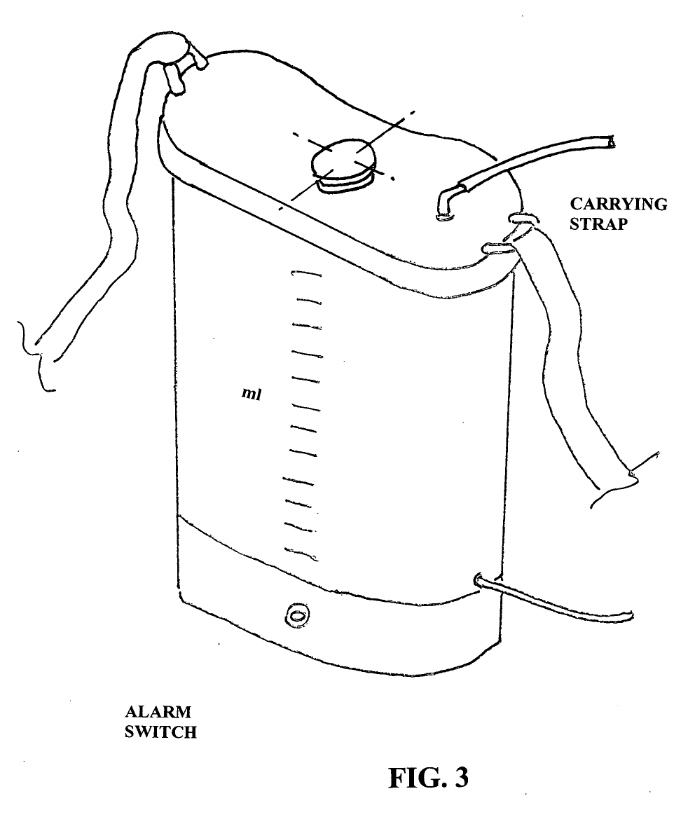

FIG. 3 is a drawing of a simple portable unit that includes the magnetic switch as described above. In this embodiment the shape of the container can be varied to fit against a person's chest or side, for ease of portability in a car or walking. Portable units without the complications of the warning elements may be preferred for their simplicity. However, as explained above, the user must exercise special care to not let the fluid level drop to the level of the exit port.

### Alternate Magnetic Float Design

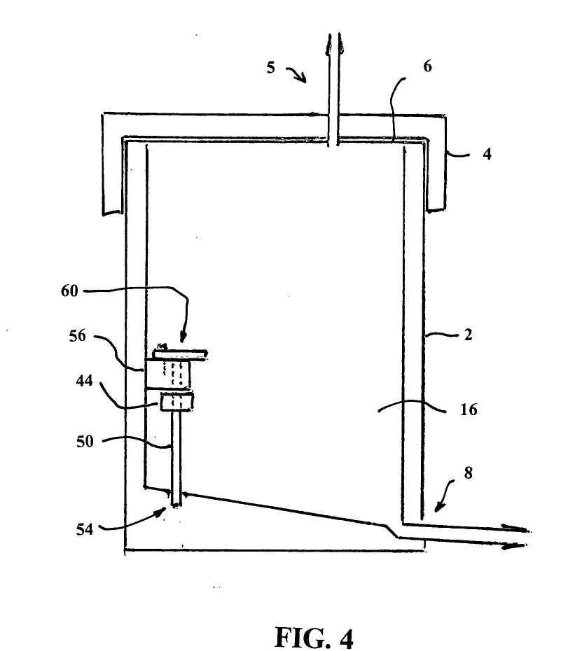

FIG. 4 shows an alternate magnetic float design. This design includes ⅛ inch diameter stainless rod 50. The lower end of rod 50 can be inserted in 5/32 inch diameter, ⅛ inch deep hole 54 in the bottom of container 2 is positioned over magnetic switch (not shown in FIG. 4 but shown in FIG. 2). Also mounted on the side of container 2 is plastic positioner 56. Hole 58 drilled through positioner 56 positions the upper portion of rod 50. Pivoting cover tab 60 covers hole 58 but can be pivoted aside to insert rod 50 through positioner 56 then through magnetic float 44 and into hole 54. Rod 50 is then captured in place by pivoting cover tab 60 over rod 50. Float 44 can only rise about 3″, it serves no purpose except to float high enough to break contact with magnetic switch. So when liquid is depleted to a certain level, it will descend and trigger the alert mechanism. The entire float mechanism can be removed for cleaning, except the plastic rod positioner 56 and cover tab 60 will remain.

### Other Considerations

Container 2 may preferably be sized to hold one to three feedings or more. Single feedings could vary from approximately 8 oz. to over 16 oz. A preferred size of container 2 is for about 28 oz, but could be sized to hold as much as 34 oz. (or more) thereby easily permitting more than one feeding per fill. After the first single feeding, the fluid container can then be placed in refrigeration until the next feeding. Preferably, container 2 is generally cylindrical and wide enough for a persons hand to be inserted to make hand cleaning easy. The removable lid 4 may contain a filling port with a threaded cap so that additional nutrition such as pulverized vitamins or medicines may be added before or after the unit is sealed.

The Applicant provides the following guidance from his own experience with prototypes of his invention: The nutritional liquid prescribed by Applicant's doctor comes in 8 oz cans. (There may be other types and sizes available that he is not aware of.) Applicant was prescribed four feedings per day, two 8 oz. cans per feeding, twice a day, and then 1½ cans each for the last two feedings, for a total of 7 cans, or 56 oz per day. For the first two feedings he only puts in 16 oz each time, and thoroughly washes the feeder and flushes the feeder tube after each feeding. He puts the contents of three 8 oz. cans in the feeder at the beginning of the last two feedings, and just feeds ½ of that at each feeding. It makes the small tube a little harder to wash thoroughly if it is not washed after each feeding. He flushes his stomach tube immediately after each and every feeding. Note that Applicant refers to the tube coming out of the feeder as the feeding tube, and the one inserted into his stomach as his stomach tube.) Of course the small plastic feeder tube coming out of the feeder is easily replaced should it become necessary.

### Variations

Preferred embodiments of the present invention have been shown in detail. However the reader should realize that many changes and variations are possible without departing from the concepts of the present invention. For example, while the second preferred embodiment has a single small LED light, the unit could use an LED rope light within the base so that the complete base would ‘glow’ when the magnetic switch is activated. Instead of the sloped bottom as shown in FIG. 4, an alternative would be to provide a sloping V-groove or a U-grove on the bottom along a diameter of the container that starts at a zero depth at one side and ends at a depth of about ¼ inch at the exit port. This approach may make the design of the magnetic alarm unit somewhat easier. In portable units the above V-groove or U-groove could be combined with the sloping bottom to make it easy to pump the container down to almost the last drop. Other types of finger or hand operated air pressure units are available such as small hand pumps for pressuring toys. These units could be easily adapted for use here in place of the flexible bulb unit. Preferably the pressure unit should be designed so that the maximum pressure easily applied is about 1.0 psi. Use of pumps capable of much higher pressures may require a relief valve set to relieve pressures higher than about 1.0 psi. The fluid container may be provided with a handle attached to two bands (not shown here) that allows either a right or left handed person to locate the air pressure bulb and the discharge tube in any orientation that is comfortable to the user. The two-band handle is free to rotate around the fluid container for complete freedom to locate the air bulb pump and liquid discharge point to any position desired for ease of operation. The other level warning units could be used such as a float adapted to produce an electrical contact when the fluid level approached the exit port. Therefore the reader should determine the scope of the present invention by the appended claims and their legal equivalence.

I claim:

1. A self-powered, enteral tube feeding device comprising:
A) a fluid container,
B) a removable lid for pressure sealing the container,
C) a fluid exit port,
D) a flexible tube providing fluid communication from the exit port of the device to a through-the-abdominal-wall port of a user,
E) finger or hand operated air pressurizer unit adapted to apply air pressure to said fluid container, and
F) a flexible tube providing air pressure communication between said finger or hand operated pressurizer unit;
wherein fluid is forced out of the container and into the patient's stomach or small intestine with air pressure provided by said finger or hand operated air pressurizer unit.

2. The feeding device as in claim 1 wherein said air pressurizer unit comprises a flexible bulb and two check valves.

3. The feeding device as in claim 1 and further comprising a fluid level warning device adapted to warn the user when fluid levels within said container is near said fluid exit port.

4. The feeding device as in claim 3 wherein said warning device comprises a magnet and a magnetic switch.

5. The feeding device as in claim 3 wherein said warning device comprises an audible alarm triggered by said magnetic switch.

6. The feeding device as in claim 3 wherein said warning device comprises a vibrating element triggered by said magnetic switch.

7. The feeding device as in claim 3 wherein said warning device comprises a warning light triggered by said magnetic switch.

8. The feeding device as in claim 4 wherein said warning device further comprises a warning element chosen from the following types of warning elements:
A) an audible element,
B) a warning light element, and
C) a vibration element.

9. The feeding device as in claim 7 wherein said warning light is an LED.

10. The feeding device as in claim 7 wherein said warning light is a rope light.

11. The feeding device as in claim 1 and further comprising a handle mounted on said container.

12. The feeding device as in claim 11 wherein said handle is adjustable to accommodate either left or right handed users.

13. The feeding device as in claim 1 wherein said container comprises a bottom surface sloping toward said exit port.

14. The feeding device as in claim 13 wherein said bottom surface comprises a V-groove at said exit port adapted to avoid air entering said exit port prior to virtually all fluid being forced out.
